(19) United States
(12) Patent Application Publication
(10) Pub. No.: US 2016/0166624 A1
(43) Pub. Date: Jun. 16, 2016

(54) ANTI-VIRAL COMPOSITIONS

(71) Applicant: FLUTRENDS INTERNATIONAL, LLC, Denver, CO (US)

(72) Inventor: Steve W. SCHWARTZ, Denver, CO (US)

(21) Appl. No.: 14/436,803

(22) PCT Filed: Oct. 17, 2013
(86) PCT No.: PCT/US13/65390

§ 371 Date: Apr. 17, 2015

Related U.S. Application Data
(60) Provisional application No. 61/716,420, filed on Oct. 19, 2012.

(51) Int. Cl. A61K 36/185; A61K 47/10; A61K 9/00; A61K 47/12; A61K 31/35; A61K 31/045

(52) U.S. Cl. CPC A61K 36/185; A61K 31/35; A61K 31/045; A61K 9/0043; A61K 9/0073; A61K 47/12; A61K 47/10

(57) ABSTRACT

Therapeutic and prophylactic compositions to treat and/or prevent common and recurrent viral infections. The compositions have broad-spectrum antiviral activities, and safe and substantially non-irritating. In one aspect, the present invention provides an antiviral composition that comprises one, two, three or all of the components selected from the group consisting of eucalyptol, menthol, and elderberry extract.

### TECHNICAL FIELD

The present invention relates to compositions having broad-spectrum antiviral activities that are safe and substantially non-irritating.

### BACKGROUND OF INVENTION

Influenza is a highly infectious acute respiratory disease that has plagued the human race since ancient times. Until the emergence of AIDS, flu or influenza virus was the last uncontrolled pandemic killer of humans. In the United States, influenza currently causes more morbidity and mortality than AIDS, and influenza is characterized by recurrent annual epidemics and periodic major worldwide pandemics. In fact, influenza has caused more deaths in the United States that all of the great wars combined.

Because of the high disease-related morbidity and mortality, direct and indirect social economic impacts of influenza are enormous. Yearly epidemics cause thousands of hospitalizations and many thousands of deaths in the United States alone. Four pandemics occurred in the last century and mathematical models predict 20-47 million illnesses would results during another pandemic (Meltzer, M I, Cox, N J and Fukuda, K. (1999) Emerg. Infect Dis 5:659-671).

The upper respiratory tract (URT) and genitalia are the routes most often used by viruses that lead to viral infection. Viral infections in the URT are best characterized by flu and common cold, and viral infections in genital areas are represented by herpes simplex infections, HIV infections, and other sexually transmitted diseases including hepatitis and those caused by Epstein-Barr viruses. Influenza viruses are a group of RNA viruses designated as types A, B, and C, with influenza A virus being the most virulent because influenza A undergoes periodic antigenic shifts.

When the influenza virus infects epithelial cells in the URT, it enters the host cells by a process of membrane fusion. This may occur at the cell plasma membrane or within the endocytic vacoular system. Upon binding to cell surface, the virus undergoes endocytosis is then delivered to endosomes. Viral replication by influenza type A and B viruses is primarily limited to the upper respiratory tract but can extend to the lower respiratory tract and cause bronchopneumonia, which can be fatal.

Influenza viral protein hemagglutinin (HA) is the major viral envelope protein. It plays an essential role in viral infection. HA is responsible for the attachment of the virus to sialic acid cell receptors on host cells and, HA mediates viral entry into target cells by triggering fusion of the viral envelope with cellular membranes. HA is also the major target for protective neutralizing antibodies produced by the host immune response.

Currently, influenza infection is controlled by vaccination and anti-viral compounds. Inactivated influenza vaccines are now in worldwide use. The vaccine viruses are grown in eggs, inactivated by chemical means and purified. The vaccines are usually trivalent, containing representative influenza A viruses (H1N1 and H3N2) and influenza B strains. The vaccine strains need to be regularly updated in order to maintain efficacy; this effort is coordinated by the World Health Organization (WHO). During inter-pandemic periods, it usually takes eight months before the updated influenza vaccines are ready for the market (Wood, J. (2001) Phil Trans R Soc Lond B 356:1953-1960). Historically however, pandemics spread to most continents within six months, and future pandemics are expected to spread even faster with increased international travel. Historically, most deaths occurring in pandemics occurred in the first four months of the spread of the virus. Therefore it is predictable that an effective vaccine will be unavailable or in very short supply during the first waves of future pandemics.

In light of these slow supply problems and logistics for vaccines, anti-viral compounds and compositions have become the only potential alternative for controlling pandemics during the initial period when vaccines are not available. Two classes of antiviral compounds are currently on the market: the M2 inhibitors, such as amantadine and rimantadine; and the NA inhibitors, which include oseltamivir (TAMIFLU™) and zanamivir (RELENZA™). Both classes of molecules have proven efficacy in prevention and treatment of influenza. However, side effects and the risk of generating drug-resistant viruses remain the top two concerns for using anti-viral compounds or compositions widely for prophylaxis of viral infection.

Thus, there is a need for novel therapeutic and prophylactic modalities to treat and/or prevent common and recurrent viral infections and to address future influenza pandemics.

### SUMMARY OF INVENTION

The present invention recognizes that current therapeutics for preventing and treating infection by viral pathogens are difficult to provide in a timely manner, and can have undesirable side effects. The present invention provides antiviral compositions for preventing and treating viral pathogen infection and methods for making and using such compositions. The compositions of the present invention are generally safe, non-irritating, and have a broad-spectrum antiviral activities.

In one aspect, the present invention provides an antiviral composition that comprises one, two, three or all of the components selected from the group consisting of eucalyptol, menthol, and elderberry extract (extract of Sambucus nigra, commonly referred to as elder, elderberry, black elder, European elder, European elderberry and European black elderberry).

In certain embodiments, the compositions of the present invention comprise at least one additional compound selected from the group consisting of poloxamer 407, xyitol, sucrose, saccharin, sorbitol, glycerin, sodium benzoate, sodium chloride, octoxynol-9, citric acid, sodium chloride, thymol, menthol, ethanol, octylphenoxypolyethoxyethanol, methyl salicylate (present as oil of wintergreen or an extract of Gaultheria procumbens) and water. Certain embodiments contain two, three, four, five or more of these additional compounds.

A specific embodiment is a composition containing eucalyptol, menthol, elderberry extract, methyl salicylate, water, ethanol, xylitol, poloxamer 407, glycerin, sorbitol, sodium chloride, citric acid, sodium benzoate, and thymol.

Another specific embodiment is a composition containing eucalyptol, elderberry extract, water, ethanol, poloxamer 407, glycerin, sorbitol, sodium saccharine, citric acid, sodium benzoate, methyl salicylate, thymol, octylphenoxypolyethoxyethanol, and menthol.

The compositions may be formulated as a solution, a paste, a gel, a suspension, a lotion, a cream, an aerosol, a dressing, a bandage, a lacquer, an ointment, or other formulation appropriate for local application for the treatment or prevention of viral infectious diseases. Such compositions are preferably formulated as liquids for nasal administration such as a spray or inhalant. Such compositions may be formulated to be isotonic. Such compositions may also be formulated to have a pH between about 2 and about 7. Certain embodiments may have a pH between 3 and 6. Certain embodiments may have a pH between 3 and 5. Certain embodiments may have a pH between 3 and 4. Certain embodiments may have a pH between 3 and 3.5. Specific embodiments may have a pH of about 3.2.

Such compositions may be packaged in and/or administered via an appropriate pharmaceutical delivery system for delivery to the upper respiratory tract of a subject, such as an inhaler, a nebulizer, an atomizer, a nasal spray bottle, or a dropper.

When administered in these formulations, including through the use of such delivery systems, these compositions may be applied to epithelial cells, including for example, respiratory epithelial cells, adenoid epithelial cells or bronchial epithelial cells of the subject being treated. Alternatively, or additionally, these compositions may be applied to endothelial cells of the subject being treated. The composition may be administered from one to four times a day or more, if indicated or needed for prevention or treatment.

In certain embodiments, the eucalyptol is present as Eucalyptus globulus oil or Eucalyptus oil.

In certain embodiments, the elderberry extract is an extract of black elderberry (Sambucus nigra).

In certain embodiments, the methyl salicylate is present as oil of wintergreen or an extract of Gaultheria procumbens.

These embodiments may also contain preservatives such as sodium benzoate and/or citric acid. Certain embodiments include citric acid, water and ethanol formulated for nasal administration as described below. Certain embodiments consist essentially of citric acid, water and ethanol formulated for nasal administration as described below. Certain embodiments consist of citric acid, water and ethanol formulated for nasal administration as described below.

In certain embodiments, the antiviral compositions of the present invention include excipients such as a pH adjusting agent, a pH buffer, a viscosity modifier, an osmotic agent, a flavor, a sweetener, a preservative, an adhesive, a thickener and a colorant.

In certain compositions of the present invention, the concentration of eucalyptol (w/v) may be about 0.01% to about 2% (e.g., about 0.01%, 0.02%, 0.03%, 0.04%, or 0.05% to about 0.5%, 1%, 1.5%, 2%), the concentration of the menthol (w/v) may be about 0.001% to about 2% (e.g., about 0.001%, 0.005%, 0.01%, 0.02%, 0.03%, 0.04%, 0.05%, 0.06%, 0.07% to about 1%, 2%), the elderberry extract (w/v) may be about 0.01% to about 2% (e.g., about 0.01%, 0.02%, 0.03%, 0.04%, or 0.05% to about 0.1%, 0.2%, 0.3%, 0.4%, 0.5%, 1%, 0.5%), and the methyl salicylate (w/v) may be about 0.01% to about 2% (e.g., about 0.01%, 0.02%, 0.03%, 0.04%, or 0.05% to about 0.1%, 0.2%, 0.3%, 0.4%, 0.5%, 1%, 1.5%, 2%. The above ranges and the other ranges disclosed in other portions of the present application may include any values there between.

The compositions of the present invention may be applied by spraying, inhaling, rubbing, spreading, dropping, cleansing, rinsing, or soaking the site of intended treatment with the antiviral compositions.

The present invention also provides kits for ameliorating viral infection that comprise the antiviral compositions described above, in a container or an applicator and instructions for administering the compositions.

In another aspect, the present invention provides methods for ameliorating viral infection. Such methods include administering to a subject (e.g., a mammal, especially a human) in need thereof the compositions of the present invention in an amount effective to ameliorate or prevent viral infection.

In certain embodiments, the viral infection is caused by a virus selected from the group consisting of influenza virus, rhinovirus, coronavirus, parainfluenza virus, or respiratory syncytical virus. The influenza virus may include the subtypes H1N1, H3N2, H5N1 or H7N9.

In certain embodiments, the viral infection results in a common cold or flu.

In certain embodiments, the antiviral compositions of the present invention are administered locally, such as to nasal membranes, skin, or oral membranes.

In other embodiments, the antiviral compositions of the present invention are administered orally.

In certain embodiments, the viral infection is in the upper respiratory tract.

This Summary of the Invention is neither intended nor should it be construed as being representative of the full extent and scope of the present invention. Moreover, references made herein to “the present invention,” or aspects thereof, should be understood to mean certain embodiments of the present invention and should not necessarily be construed as limiting all embodiments to a particular description. The present invention is set forth in various levels of detail in the Summary of the Invention as well as in the attached drawings and the Description of Embodiments and no limitation as to the scope of the present invention is intended by either the inclusion or non-inclusion of elements, components, etc. in this Summary of the Invention. Additional aspects of the present invention will become more readily apparent from the Description of Embodiments, particularly when taken together with the drawings.

### BRIEF DESCRIPTION OF DRAWINGS

FIG. 1 is a schematic diagram of the virucidal suspension efficacy testing protocol used in the efficacy testing described in the Examples section of this disclosure.

### DESCRIPTION OF EMBODIMENTS

### Antiviral Compositions

The present invention provides antiviral compositions. “Anti-viral” refers to the capability of reducing the number of viral particles in an infected subject (e.g., a cell line, a person or an animal) and/or reducing the likelihood of a subject exposed to potentially infective viral particles to contract a viral disease (i.e., preventing viral infection). In other words, the number of viral particles that infect a subject, or the likelihood of a subject to be infected by viral particles, is significantly reduced with the administration of an antiviral compound or composition compared to that without the administration of the antiviral compound or composition. In certain embodiments, the antiviral compound or composition inhibits or reduces the contact between the viral particles and the subject, and/or the replication or emission of the viral particles.

The present invention, in certain embodiments, provides compositions that comprise eucalyptol, menthol, elderberry extract, and methyl salicylate. Such compositions possess superior anti-viral activity to prior art formulations.

In certain embodiments, these antiviral compositions comprise one or more of poloxamer 407, xyitol, sucrose, saccharin, sorbitol, glycerin, sodium benzoate, octoxynol-9, citric acid, sodium chloride, thymol, ethanol, and water.

In certain embodiments, the antiviral compositions do not further comprise any active antiviral ingredient other than one or more of eucalyptol, menthol, elderberry extract, and methyl salicylate. An “active antiviral ingredient” refers to a compound that has an antiviral activity when administered individually or in combination of one or more other compounds that do not have any antiviral activity. “Antiviral activity” refers to the capability of reducing the number of viral particles in an infected subject and/or reducing the likelihood of a subject exposed to potentially infective viral particles to contract a viral disease.

In certain embodiments, the compositions of the present invention comprise components that have not been known as having antiviral activities, and thus were previously regarded as “inactive ingredients” or “pharmaceutical excipients.” Such “inactive ingredients” refer to compounds that are included in antiviral compositions, but do not have antiviral activities.

“Pharmaceutical excipients” refers to compounds that are included in antiviral compositions, and are pharmaceutically acceptable, but are typically not used as an active antiviral ingredient.

“Pharmaceutically acceptable” refers to the property of a compound that is within the scope of sound medical judgment, suitable for use in contact with the tissues of humans and lower animals without undue toxicity, irritation, allergic response, and the like.

In certain embodiments, the formulations of the present invention are safe, substantially non-irritating, and of a broad spectrum of antiviral activity. As used herein, “safe” refers to the property of a composition (or a compound) that is substantially free of systemic toxicity; “non-irritating” refers to the property of a composition (or a compound) that causes no or an acceptably low level reaction in the area of application; and “broad-spectrum anti-viral activity” refers to the ability of a composition (or a compound) to inhibit or reduce the infectivity of more than one strain type of virus.

### Antiviral Components

Eucalyptol or Eucalyptus, is also known as 1,8-cineol, 1,8-cineole, limonene oxide, cajeputol, 1,8-epoxy-p-menthane, 1,8-oxido-p-menthane, eucalyptol, eucalyptole, 1,3,3-trimethyl-2-oxabicyclo[2,2,2]octane, cineol, cineole. Eucalyptol forms the dominant portion of oil collected from the Eucalyptus genus of plants and particularly, Eucalyptus globulus. The compound is widely available from commercial sources, and may also be used in the form of a tincture prepared from elderberry plant leaves or flowers (Eucalyptus globules) available from homeopathic supply sources (HPUS Eucalyptus Globulus Mother Tincture).

Menthol is an organic crystalline substance, clear or white in color, solid at room temperature, but melts slightly above room temperature. The main form of menthol occurring in nature is (−)-menthol (the 1R,2S,5R configuration). Menthol is made synthetically or obtained from peppermint or other mint oils, and may be purchased commercially, or is made synthetically and may also be used in the form of peppermint oil, or as essential oil of Mentha, available from homeopathic supply sources (HPUS Mentholum).

Elderberry extract is a plant extract derived from any parts of Sambucus nigra plants (commonly called Elder, Elderberry, Black Elder, European Elder, European Elderberry, European Black Elderberry, Common Elder, or Elder Bush). The extract may be derived from the stem, bark, leaves, flowers, fruits, and root of the plant. The extract is available commercially and may also be used as a source supplied as a tincture made principally from leaves and flowers, available from homeopathic supply sources (HPUS Sambucus Nigrans Mother Tincture).

### Additional Composition Components

The compositions of the present invention may optionally comprise one or more useful excipients, including but not limited to, pH adjusting agents, pH buffering agents, viscosity modifiers, osmotic agents, flavors, sweeteners, preservatives (e.g., metal chelators), adhesives and colorants. The selection and use of such agents are determined based on practices known to those skilled in art.

Methyl salicylate, also referred to as methyl 2-hydroxybenzoate, is produced by many plants, particularly Gaultheria procumbens (referred to an Eastern teaberry or wintergreen), and the distillate of these plants is commonly referred to as oil of wintergreen or wintergreen oil. Methyl salicylate is produced by esterifying salicylic acid with methanol, and is available commercially in pure form or as tinctures or oils from homeopathic supply sources (HPUS Gaultheria Mother Tincture), and may be used to impart flavor to compositions of the invention.

Thymol (also known as 2-isopropyl-5-methylphenol) is a monoterpene phenol derivative of cymene, found in oil of thyme, that may be extracted from Thymus vulgaris (common thyme) and various other kinds of plants as a white crystalline substance that may be used to impart flavor to compositions of the invention.

Additional components that may be present in the compositions of the invention include ethanol, citric acid to adjust pH, sorbitol to adjust the taste and/or texture of the formulations, glycerin to adjust the texture of the formulations, poloxamer 407 to adjust the viscosity of the formulations, octoxynol-9 as a surfactant in the formulations, xylitol as a sweetener in the formulations, saccharin as a sweetener in the formulations, and sodium benzoate as a preservative in the formulations.

Exemplary Antiviral Compositions of the present invention include, but are not limited to, the following compositions:

### Antiviral Composition 1

| Material | | Quantity | |
|---|---|---|---|
| HPUS Sambucus Nigrans | 1X (BOIRON ™) | 5 | liters |
| MOTHER TINTURE | | | |
| HPUS Eucalyptol Oil | 1C (=2X) | 5 | liters |
| | (BOIRON ™) | | |
| HPUS Eucalyptus Globulus | 1X (BOIRON ™) | 500 | ml |
| MOTHER TINCTURE | | | |
| HPUS Gaultheria | 1X (BOIRON ™) | 500 | ml |
| MOTHER TINCTURE | | | |
| HPUS Mentholum (Crystals) | 1C (=2X) | 500 | ml |
| | (BOIRON ™) | | |
| Poloxamer 407 | | 6000 | grams |
| Sorbitol | | 3500 | grams |
| Glycerin | | 5620 | grams |
| Xylitol | | 10 | kilograms |
| Sodium Chloride | | 2185 | grams |
| Citric Acid, anhydrous | | 1250 | grams |
| Sodium Benzoate | | 500 | grams |
| Ethanol (190 Proof) | | 24.37 | liters |
| Thymol | | 150.0 | grams |
| Water | | QS | |
| | Total Quantity: | 250 | liters |

### Antiviral Composition 2

| Material | Percent (w/w) | Quantity | |
|---|---|---|---|
| Sambucus Nigrans | 0.983 | 3.3 | lbs |
| Eucalyptol | 0.0107 | 16 | grams |
| Eucalyptus Globulus | 0.0090 | 14 | grams |
| Gaultheria | 0.0090 | 14 | grams |
| Menthol Crystal | 0.0012 | 2.0 | grams |
| Poloxamer 407 | 2.38 | 8.0 | lbs |
| Sorbitol | 0.6977 | 2.3 | lbs |
| Glycerin | 1.1179 | 3.7 | lbs |
| Xylitol | 1.982 | 6.6 | lbs |
| Sodium Chloride | 0.436 | 1.5 | lbs |
| Citric Acid, anhydrous | 0.246 | 371 | grams |
| Sodium Benzoate | 0.103 | 155 | grams |
| Ethanol (95%) | | 56.35 | lbs |
| Thymol | 0.0293 | 44.0 | grams |

| | |
|---|---|
| Water | QS |

| | | |
|---|---|---|
| Total Quantity: | 40 | gallons |

The compositions of the present invention may be generally prepared by first dissolving appropriate amounts of various components (e.g., poloxamer 407, sorbitol, glycerin, xylitol and/or saccharin, sodium benzoate, elderberry extract, citric acid) in water, optionally adjusting pH to facilitate the dissolution of the components, filtering the solution with an appropriate membrane pore size, adjusting the pH to the target pH range if needed, and adding water to the final weight.

Separately, appropriate amounts of various components (e.g., eucalyptol, methyl salicylate, menthol, thymol, octoxynol) may be dissolved in ethanol. This solution may also optionally be treated to adjust pH to facilitate the dissolution of the components, filtering the solution with an appropriate membrane pore size, adjusting the pH to the target pH range if needed, and adding ethanol to the final weight.

The non-aqueous phase is slowly added to the aqueous phase with continual mixing, and final pH measurement is taken to adjust final pH to between pH 2 and pH 7. The compositions may be further sterilized (e.g., by autoclaving), and stored in appropriate containers.

An exemplary method for preparing the antiviral compositions of the invention may include the following general steps:

A. Prepare aqueous phase by:

Adding purified water to a clean preparation container, and begin mixing the water with an over-head mixer at a speed sufficient to create a vortex;

Slowly add the Poloxamer 407 into the vortex of the purified water and continue mixing until dissolved;

Add sorbitol to the mixing Poloxamer/purified water solution and continue mixing until dissolved;

Add glycerin to the Poloxamer/Sorbitol/purified water solution and continue mixing until dispersed;

Add saccharin or xylitol to the solution and continue mixing until dissolved;

Add sodium benzoate to the solution from the previous step and continue mixing until dissolved;

Add elderberry extract to the solution from the previous step and continue mixing until dissolved;

Add citric acid to the solution from the previous step and continue mixing until dissolved.

B. Prepare non-aqueous phase by:

Adding ethanol to a covered mixing container and begin mixing the ethanol in the covered container;

Add eucalyptol to the ethanol and continue mixing until dispersed;

Add methyl salicylate to the solution from the previous step and continue mixing until dispersed;

Add menthol to the solution from the previous step and continue mixing until dissolved.

Add thymol to the solution from the previous step and continue mixing until dissolved.

Add oxtoxynol to the solution from the previous step and continue mixing until dissolved.

C. Combine aqueous phase and non-aqueous phase:

Add the non-aqueous phase to the aqueous phase while continually mixing, and qs to final volume with water. Remove an aliquot of the final volume of solution and measure and record the pH. The pH of the solution may be adjusted to about pH 3.2 with suitable acidic, basic or buffering compounds/solutions.

Another exemplary method for preparing the antiviral compositions of the invention includes the following steps:

A. Prepare aqueous phase by:

225.63 liters of Purified Water, USP to an overhead mixer and run at a rate sufficient for a deep vortex. Slowly add 6000 grams of Poloxamer 407 into the vortex. Add 3500 grams of Sorbitol. Add 5620 grams Glycerin. Add 10,000 grams of Xylitol, NF. Add 500 grams of Sodium Benzoate. Add 1250 grams of Citric Acid Anhydrous. Add 2185 grams of Sodium Chloride. Continuously mix for 15-20 minutes.

In a separate suitable container (approximately 30 L) add 24.37 Liters of 95% Ethanol 190 Proof USP. Begin mixing and add 150 grams of Thymol, NF. Continue to mix until all Thymol has dissolved. Visually confirm all poloxamer is dissolved in aqueous phase in large tank. Combine non-aqueous phase into aqueous phase by pouring non-aqueous phase into vortex in large aqueous phase tank. Pull sample for measurement of pH. Adjust the pH to 3.2 (+/−) 0.1 using 2N HCL. QS the final volume to 250 L with Purified Water, USP. Label “Stock Solution.”

B. Prepare HPUS #1Sumbucus Nigrans solution:

Mix 45 liters of 25% volume Ethanol in purified water, USP with 5000 ml HPUS Sambucus nigrans 1× (sterilized by passing through a 5-micron filter), mix and label HPUS #1Sumbucus Nigrans solution.

### C. Prepare HPUS #2 Eucalyptol USP:

Mix 5940 cc of 95% Ethanol, NF at speed sufficient for a deep vortex. Slowly add 60 cc of Eucalyptol oil, USP. Add 45 L of 35% vol Ethanol, NF with 5000 cc of Eucalyptol oil/Ethanol solution and label HPUS #2 Eucalyptol USP.

### D. Prepare HPUS #3 Eucalyptus Globulus:

Mix 4500 cc of 35% Ethanol, NF at speed sufficient for a deep vortex, with 500 cc of MOTHER TINCTURE Eucalyptus Globulus 1×. Mix with 45 L of 20% vol Ethanol, NF, and label HPUS #3 Eucalyptus Globulus.

### E. Prepare HUPS #4 Gaultheria (Methyl Salicylate):

Mix 4500 cc of 35% Ethanol, NF with 500 cc of MOTHER TINCTURE Gaultheria 1×. Mix with 45 L of 20% vol Ethanol, NF, and label HPUS #4 Gaultheria.

### F. Prepare HUPS #5 Mentholum:

Mix 594 cc of 95% Ethanol, NF at speed sufficient for a deep vortex. Slowly add 6.0 grams of Mentholum crystals (USP Menthol crystals) until fully dissolved. Add 500 cc of the resulting solution to 4500 cc of 65% vol Ethanol, NF. Add 45 L of 20% vol Ethanol, NF and label as HPUS #5 Mentholum.

G. Prepare HPUS combination formula:

Mix 50 liters HPUS #3 Eucalyptus Globulus with 50 liters of HPUS #1 Sambucus Nigrans and with 50 liters of HPUS #4 Gaultheria and with 50 liters of HPUS #5 Mentholum and with 50 liters of HPUS #2 Eucalyptol. Add 250 L of Stock Solution. QS final volume to 500 L with Purified Water USP. Mix for 20 minutes, and pull sample for measurement of pH. Adjust the solutions pH to 3.2 (+/−) 0.1 using 2N HCL.

### Use of Antiviral Compositions

The present invention also provides a method for ameliorating viral infection comprising administering to a subject in need thereof the compositions described herein in an amount effective to ameliorate viral infection. Ameliorating viral infection is understood to encompass (1) reducing or eliminating the likelihood that a person or an animal exposed to potentially infective viral particles will be infected with the viral particles, or (2) reducing or eliminating the progression of viral infection (e.g., reducing the number of viral particles in a host). A “subject in need thereof” may be a human or an animal (e.g., a mammal) that is at risk for developing viral infection (e.g., being exposed to potentially infective viral particles) or already has contracted a viral infection.

The compositions of the present invention may be administered to a subject locally. The term “local” encompasses application in and around the site of intended treatment, and excludes peroral, subcutaneous, intravenous and intramuscular administration, which are categorized as systemic administration. Exemplary local administration includes, but is not limited to: (1) “topical” application, including the treatment on the human skin, hair, and nail; and (2) “mucosal” application, including the treatment on the nasal mucous membrane, oral mucous membrane (also referred to as oral cavity).

The compositions of the present invention may be in any form suitable for local administration. For example, the composition may be in a form of a solution, paste, gel, suspension, lotion, cream, aerosol, dressing, bandage, lacquer, or ointment formulation for local application. In certain embodiments, the compositions of the present invention are formulated (or adapted) for preventing or treating virus infection through nasal passages, such as in a form of nasal ointments, nasal drops, nasal washes, nasal packings, inhalants or nasal sprays.

Any methods appropriate for local administration of a composition to a subject known in the art may be used in the present invention. Such methods generally cause the formulation to coat and remain in contact with those membranes or skin surfaces for a period of time, like a chemical barrier at those sites.

One exemplary method of applying an antiviral formulation of the present invention to the nasal mucus membrane, the oral cavity, or the like involves removing a small quantity (such as several milliliters) of a solution, gel, suspension, lotion, cream, ointment, or similar formulation from a container, followed by spraying or by squeezing the container which is preset for a desired amount directly at the area(s) of interest, or by spreading the formulation across the mucus or skin area(s) with a finger or an applicator.

The compositions of the present invention may be used to ameliorate various viral infections such as infection of influenza virus, rhinovirus, corona virus, and respiratory syncytical virus. They may be useful in preventing, reducing the duration, or relieving, the symptoms of the common cold, flu, and other respiratory viral infections.

The effectiveness of a given antiviral composition according to the present invention may be evaluated using in vitro cultured virus-transfected cells (such as those described in the examples below).

Another embodiment of the invention relates to the use of any of the antiviral compositions described herein in the preparation of a medicament for the treatment or prevention of viral infection.

The invention now being generally described will be more readily understood by reference to the following examples, which are included merely for the purposes of illustration of certain aspects of the embodiments of the present invention. The examples are not intended to limit the invention, as one of skill in the art would recognize from the above teachings and the following examples that other techniques and methods can satisfy the claims and can be employed without departing from the scope of the claimed invention.

### EXAMPLES

### Example 1

### Virucidal Suspension Efficacy Test: Respiratory Syncytial Virus (RSV)

This study was designed to measure virucidal effectiveness of a test composition of the present invention. It determines the potential of the test composition to kill RSV virus in suspension. The test follows the principle outlined in the American Society for Test Materials (ASTM) test method designated E 1052-96 “Standard Test Method for Efficacy of Antimicrobial Agents against Viruses in Suspension.”

The test compositions are evaluated against the challenge virus in suspension. Two test compositions, one lot each, are evaluated for inactivation of Respiratory Syncytial Virus at one exposure (contact) time. One replicate run is performed for each condition. To minimize buffer interference and to minimize reduction of virucidal activity, the volume of virus inoculum added to test material is kept to equal or less than 10% of the total volume of the test. Aliquots are removed at the completion of the contact time from the test composition/virus reaction mixture; neutralized (quenched); and inoculated onto the appropriate host cell system. The inoculated host system is incubated and read for presence of infectious virus.

### Composition Tested: Antiviral Composition 1

| Material | | Quantity | |
|---|---|---|---|
| HPUS Sambucus Nigrans | 1X (BOIRON ™) | 5 | liters |
| MOTHER TINTURE | | | |
| HPUS Eucalyptol Oil | 1C (=2X) | 5 | liters |
| | (BOIRON ™) | | |
| HPUS Eucalyptus Globulus | 1X (BOIRON ™) | 500 | ml |
| MOTHER TINCTURE | | | |
| HPUS Gaultheria | 1X (BOIRON ™) | 500 | ml |
| MOTHER TINCTURE | | | |
| HPUS Mentholum (Crystals) | 1C (=2X) | 500 | ml |
| | (BOIRON ™) | | |
| Poloxamer 407 | | 6000 | grams |
| Sorbitol | | 3500 | grams |
| Glycerin | | 5620 | grams |
| Xylitol | | 10 | kilograms |
| Sodium Chloride | | 2185 | grams |
| Citric Acid, anhydrous | | 1250 | grams |
| Sodium Benzoate | | 500 | grams |
| Ethanol (190 Proof) | | 24.37 | liters |
| Thymol | | 150.0 | grams |
| Water | | QS | |
| | Total Quantity: | 250 | liters |

The challenge virus for this study: Respiratory Syncytial Virus

The host cell line: HeLa cells

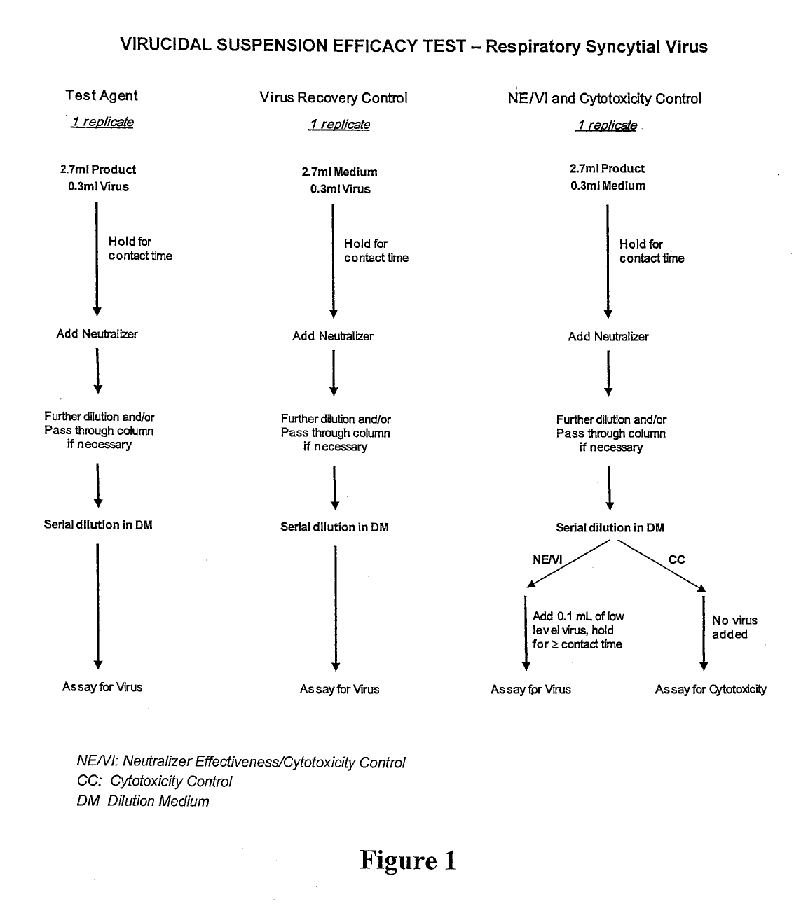

The experimental design is required as part of good laboratory practices (GLP) regulations. The study flow diagram is summarized in FIG. 1, with details described below.

### Inoculum Preparation:

Viral stocks purchased from reputable sources are propagated. They are titered and stored in an ultra-low temperature freezer. Records are maintained that demonstrate the origin of the virus. Frozen viral stocks are thawed on the day of the test (fresh stock cultures may be used at the discretion of the Study Director).

Test: Two compositions, one lot each, are evaluated at one exposure (contact) time. One replicate run is performed for each condition. For each replicate run, a 2.7-mL aliquot of the composition is spiked with 0.3 mL of the virus suspension and mixed thoroughly by vortexing. At the completion of each contact time, an aliquot of the reaction mixture is pulled and immediately mixed with an equal volume of neutralizer. The neutralized sample is further quenched by dilution with dilution medium and/or passing through a gel-filtration Sephacryl column to remove cytotoxicity. The quenched sample is then serially ten-fold diluted with dilution medium and selected dilutions inoculated onto host cells to assay for infectious virus. Where columns are used, each sample is loaded into separate pre-spun Sephacryl columns. The eluates are aseptically collected and serially diluted in ten-fold increments. If columns are not used, serial ten-fold dilutions of neutralized virus-test composition mixture are prepared in appropriate diluent.

Infectivity Assay:

The residual infectious virus in the test and controls is detected by viral-induced cytopathic effect (CPE). Selected dilutions of the neutralized inoculum/test composition mixture are added to cultured cell monolayers at a minimum of four wells per dilution per sample. The host cells are washed twice with phosphate buffered saline (PBS) prior to inoculation. The inoculated plates are incubated at 36±2° C. in 5±1% CO₂ for 14-18 days. The host cell cultures are observed and re-fed, as necessary, during the incubation period. These activities, if applicable, are recorded. Then the host cells are examined for presence of infectious virus. The resulting virus-specific CPE and test composition-specific cytotoxic effects are scored by examining both test and controls.

Controls:

1. Neutralizer Effectiveness/Viral Interference Control:

This control determines if residual active ingredient is present after neutralization and if the neutralized test composition interferes with virus infectivity. This control is performed for each of the test compositions individually.

A 2.7-mL aliquot of each composition is mixed thoroughly with 0.3 mL of medium in lieu of the challenge virus) by vortexing, holding for contact time, and then neutralizing by adding equal volume of neutralizer. The neutralized sample may be further quenched by dilution with dilution medium and/or passing through a gel-filtration Sephacryl column to remove cytotoxicity, if such procedure is used for the test composition runs.

The neutralized and quenched sample is then serially tenfold diluted using dilution medium. Each dilution is divided into two portions, one for Neutralizer effectiveness/viral interference control, and the other for cytotoxicity control. For the Neutralizer effectiveness/viral interference control, 100 μL of a low titered (10⁻² to 10⁻³) virus stock is added to 4.5 mL of selected dilutions of the solution and held for a period equivalent to, or greater than, the longest contact time. The virus-spiked solution is used to inoculate host cells as described for the test procedure.

2. Cytotoxicity Control:

This control is performed for each of the test compositions individually. Selected dilutions of the sample obtained from the Neutralizer effectiveness/viral interference control run are inoculated onto host cells and incubated together with other test and control samples as described for the test procedure. The condition of the host cells is recorded at the end of the incubation period. The cytotoxic effects should be distinct from virus-specific cytopathic effects, which are evident in the stock titer and virus recovery control cultures.

3. Virus Recovery Control:

A 2.7-mL aliquot of medium (in lieu of the test composition) is mixed thoroughly with 0.3 mL of the challenge virus by vortexing, holding for contact time, and then neutralizing by adding equal volume of neutralizer. The neutralized sample may be further quenched by dilution with dilution medium and/or passing through a gel-filtration Sephacryl column to remove cytotoxicity, if such procedure is used for the test composition runs. The quenched sample is then serially tenfold diluted with dilution medium and selected dilutions are inoculated onto host cells to assay for infectious virus. The virus control results from this control are used as the input viral load and compared with the test composition treatment results to evaluate viral reduction by the test composition.

4. Column Titer Control (Performed Only if a Sephacryl Column is Used):

This control is performed to determine any affect the columns have on infectious virus titer. The sample for this control is acquired from a portion of the PRC, prior to passing through the columns and serially diluted in CCM, then processed in the same manner as the test.

5. Cell Viability Control:

At least four wells are inoculated with an appropriate media during the incubation phase of the study. This control demonstrates that cells remain viable throughout the course of the assay period. In addition, it confirms the sterility of the media employed throughout the assay period.

6. Virus Stock Titer Control (VST)

An aliquot of the virus used in the study (RSV) is directly serially diluted and inoculated onto the host cells to confirm the titer of the stock virus. This control demonstrates that the titer of the stock virus is appropriate for use and that the viral infectivity assay is performed appropriately.

### Calculation:

The 50% tissue culture infective dose per mL (TCID50/mL) is determined using the method of Spearman-Karber (Kärber G. Arch. Exp. Pathol. Pharmakol. Vol. 162. Pages: 480-483, 1931) or other appropriate methods such as Reed and Muench, Am. J. of Hyg. 1938, 27:493. In the case where a sample contains no detectable virus, a statistical analysis may be performed based on Poisson distribution (International Conference On Harmonization (ICH) Topic Q5A, Pages: 24-25, 1999) to determine the theoretical maximum possible titer for that sample. These analyses will be described in detail in the final report. The test results are reported as the reduction of the virus titer due to treatment with test composition expressed as log 10.

The tests are acceptable for evaluation of the test results if:

a. Virus must be recovered from the neutralizer effectiveness/viral interference control (not exhibiting cytotoxicity).

b. Viral-induced CPE must be distinguishable from test composition induced toxicity.

c. Cell Viability Control must not exhibit viral-induced CPE or cytotoxicity.

### Results

The results are shown in tables 1-3 below.

TABLE 1
Titer Results - Respiratory syncytial virus (RSV)

| | Contact | Titer ± 95% CI | Volume | Volume | Viral Load |
|---|---|---|---|---|---|
| Test Agent | Time | (Log₁₀TCID₅₀/mL) | (mL) | Correction^{a} | (Log₁₀TCID₅₀) |
| Virus Stock Titer Control | NA | 7.68 ± 0.20 | — | — | — |
| Virus Recovery Control | 5 Minutes | 6.55 ± 0.22 | 3 | 2 | 7.33 ± 0.22 |
| Cell Viability Control | NA | No virus was detected, cells remained viable; media were sterile | | | |
| Composition tested | 5 Minutes | 1.83* | 3 | 2 | ≦2.61 |
| (Lot. No. 526) | | | | | |
| Composition tested | 5 Minutes | 1.83* | 3 | 2 | ≦2.61 |
| (Lot. No. 704) | | | | | |

^{a}Volume correction accounts for the neutralization of the sample post contact time.
*No virus was detected; the theoretical titer was determined based on the Poisson distribution.

TABLE 2
Neutralizer Effectiveness and Cytotoxicity Related Controls - RSV

| | Neutralizer Effectiveness/ | Cytotoxicity |
|---|---|---|
| Dilution** | Viral Interference Control | Control |
| | Composition tested (Lot No. 526) | |
| 10⁻¹ | virus detected in all eight wells | no cytotoxicity observed in |
| | | all 8 wells |
| 10⁻² | virus detected in all eight wells | no cytotoxicity observed in |
| | | all 8 wells |
| 10⁻³ | virus detected in all eight wells | no cytotoxicity observed in |
| | | all 8 wells |
| | Composition tested (Lot No. 704) | |
| 10⁻¹ | virus detected in all eight wells | no cytotoxicity observed in |
| | | all 8 wells |
| 10⁻² | virus detected in all eight wells | no cytotoxicity observed in |
| | | all 8 wells |
| 10⁻³ | virus detected in all eight wells | no cytotoxicity observed in |
| | | all 8 wells |

**Dilution refers to the dilution ration from the post-neutralized sample

TABLE 3
Neutralizer Effectiveness and Cytotoxicity Related Controls - RSV

| | Initial Load | Output Load | Viral Reduction |
|---|---|---|---|
| Test Agent | (Log₁₀TCID₅₀) | (Log₁₀TCID₅₀) | (Log₁₀TCID₅₀) |
| Composition tested | 7.33 ± 0.22 | ≦2.61 | ≧4.72 ± 0.22 |
| (Lot No. 526) | | | |
| Composition tested | | ≦2.61 | ≧4.72 ± 0.22 |
| (Lot No. 704) | | | |

### Conclusions:

Compositions of the present invention were evaluated for the ability to inactivate Respiratory syncytial virus (RSV). Test personnel performed the inactivation procedure using RSV to spike the test agent solution. Samples were titrated by 50% tissue culture infectious dose (TCID50) endpoint assay using HeLa cells. When tested as described above, compositions of the invention inactivated RSV when the challenge virus was exposed to the test agents for 5 minutes at 20° C. Table 3 reports the individual Log 10 virus reduction factor for the test agent treatment procedure. All of the controls met the criteria for a valid test. These conclusions are based on observed data.

### Example 2

### Virucidal Suspension Efficacy Test: Human Coronavirus

This study was designed to measure virucidal effectiveness of a test composition of the present invention. The test determines the potential of the test composition to kill Coronavirus in suspension. The test follows the principle outlined in the American Society for Test Materials (ASTM) test method designated E 1052-96 “Standard Test Method for Efficacy of Antimicrobial Agents against Viruses in Suspension.”

The test compositions are evaluated against the challenge virus in suspension. Two test compositions, one lot each, are evaluated for inactivation of Human Coronavirus at one exposure (contact) time. One replicate run is performed for each condition. To minimize buffer interference and to minimize reduction of virucidal activity, the volume of virus inoculum added to test material is kept to equal or less than 10% of the total volume of the test. Aliquots are removed at the completion of the contact time from the test composition/virus reaction mixture; neutralized (quenched); and inoculated onto the appropriate host cell system. The inoculated host system is incubated and read for presence of infectious virus.

### Composition Tested: Antiviral Composition 1:

| Material | | Quantity | |
|---|---|---|---|
| HPUS Sambucus Nigrans | 1X (BOIRON ™) | 5 | liters |
| MOTHER TINTURE | | | |
| HPUS Eucalyptol Oil | 1C (=2X) | 5 | liters |
| | (BOIRON ™) | | |
| HPUS Eucalyptus Globulus | 1X (BOIRON ™) | 500 | ml |
| MOTHER TINCTURE | | | |
| HPUS Gaultheria | 1X (BOIRON ™) | 500 | ml |
| MOTHER TINCTURE | | | |
| HPUS Mentholum (Crystals) | 1C (=2X) | 500 | ml |
| | (BOIRON ™) | | |
| Poloxamer 407 | | 6000 | grams |
| Sorbitol | | 3500 | grams |
| Glycerin | | 5620 | grams |
| Xylitol | | 10 | kilograms |
| Sodium Chloride | | 2185 | grams |
| Citric Acid, anhydrous | | 1250 | grams |
| Sodium Benzoate | | 500 | grams |
| Ethanol (190 Proof) | | 24.37 | liters |
| Thymol | | 150.0 | grams |
| Water | | QS | |
| | Total Quantity: | 250 | liters |

The challenge virus for this study: Human Coronavirus

The host cell line: MRC-5 cells

The experimental designs are required as part of GLP regulations. The study flow diagram is summarized in FIG. 1, with details described below.

### Inoculum Preparation:

Viral stocks purchased from reputable sources have been propagated. They are titered and stored in an ultra-low temperature freezer. Records are maintained that demonstrate the origin of the virus. Frozen viral stocks are thawed on the day of the test (fresh stock cultures may be used at the discretion of the Study Director).

### Test:

Two compositions, one lot each, are evaluated at one exposure (contact) time. One replicate run is performed for each condition. For each replicate run, a 2.7-mL aliquot of the composition is spiked with 0.3 mL of the virus suspension and mixed thoroughly by vortexing. At the completion of each contact time, an aliquot of the reaction mixture is pulled and immediately mixed with an equal volume of neutralizer. The neutralized sample is further quenched by dilution with dilution medium and/or passing through a gel-filtration Sephacryl column to remove cytotoxicity. The quenched sample is then serially ten-fold diluted with dilution medium and selected dilutions to be inoculated onto host cells to assay for infectious virus. Where columns are used, each sample is loaded into separate pre-spun Sephacryl columns. The eluates are aseptically collected and serially diluted in ten-fold increments. If columns are not used, serial ten-fold dilutions of neutralized virus-test composition mixture are prepared in appropriate diluent.

### Infectivity Assay:

The residual infectious virus in the test and controls is detected by viral-induced cytopathic effect (CPE). Selected dilutions of the neutralized inoculum/test composition mixture are added to cultured cell monolayers at a minimum of four wells per dilution per sample. The host cells are washed twice with phosphate buffered saline (PBS) prior to inoculation. The inoculated plates are incubated at 33±2° C. in 5±1% CO₂ for 14-18 days. The host cell cultures are observed and re-fed, as necessary, during the incubation period. These activities, if applicable, are recorded. Then the host cells are examined for presence of infectious virus. The resulting virus-specific CPE and test composition-specific cytotoxic effects are scored by examining both test and controls.

### Controls:

1. Neutralizer Effectiveness/Viral Interference Control:

This control determines if residual active ingredient is present after neutralization and if the neutralized test composition interferes with virus infectivity. This control is performed for each of the test compositions individually.

A 2.7-mL aliquot of each composition is mixed thoroughly with 0.3 mL of medium in lieu of the challenge virus) by vortexing, held for contact time, and then neutralized by adding equal volume of neutralizer. The neutralized sample may be further quenched by dilution with dilution medium and/or passing through a gel-filtration Sephacryl column to remove cytotoxicity, if such procedure is used for the test composition runs.

The neutralized and quenched sample is then serially ten-fold diluted using dilution medium. Each dilution is divided into two portions, one for neutralizer effectiveness/viral interference control, and the other for cytotoxicity control. For the neutralizer effectiveness/viral interference control, 100 μL of a low titered (10⁻² to 10⁻³) virus stock is added to 4.5 mL of selected dilutions of the solution and held for a period equivalent to or greater than, the longest contact time. The virus-spiked solution is used to inoculate host cells as described for the test procedure.

2. Cytotoxicity Control:

This control is performed for each of the test compositions individually. Selected dilutions of the sample obtained from the neutralizer effectiveness/viral interference control run are inoculated onto host cells and incubated together with other test and control samples as described for the test procedure. The condition of the host cells is recorded at the end of the incubation period. The cytotoxic effects should be distinct from virus-specific cytopathic effects, which are evident in the stock titer and virus recovery control cultures.

3. Virus Recovery Control:

A 2.7-mL aliquot of medium (in lieu of the test composition) is mixed thoroughly with 0.3 mL of the challenge virus by vortexing, held for contact time, and then neutralized by adding equal volume of neutralizer. The neutralized sample may be further quenched by dilution with dilution medium and/or passing through a gel-filtration Sephacryl column to remove cytotoxicity, if such procedure is used for the test composition runs. The quenched sample is then serially ten-fold diluted with dilution medium and selected dilutions are inoculated onto host cells to assay for infectious virus. The virus control results from this control are used as the input viral load and compared with the test composition treatment results to evaluate viral reduction by the test composition.

4. Column Titer Control (Performed Only if a Sephacryl Column is Used):

This control is performed to determine any affect the columns have on infectious virus titer. The sample for this control is acquired from a portion of the PRC, prior to passing through the columns and serially diluted in CCM, then processed in the same manner as the test.

This control is performed to determine any affect the columns have on infectious virus titer. The sample for this control is acquired from a portion of the PRC, prior to passing through the columns and serially diluted in CCM, then processed in the same manner as the test.

5. Cell Viability Control:

At least four wells are inoculated with an appropriate media during the incubation phase of the study. This control demonstrates that cells remain viable throughout the course of the assay period. In addition, it confirms the sterility of the media employed throughout the assay period.

6. Virus Stock Titer Control (VST)

An aliquot of the virus used in the study (RSV) is directly serially diluted and inoculated onto the host cells to confirm the titer of the stock virus. This control demonstrates that the titer of the stock virus is appropriate for use and that the viral infectivity assay is performed appropriately.

### Calculation:

The 50% tissue culture infective dose per mL (TCID50/mL) is determined using the method of Spearman-Karber (Kärber G. Arch. Exp. Pathol. Pharmakol. Vol. 162. Pages: 480-483, 1931) or other appropriate methods such as Reed and Muench, Am. J. of Hyg. 1938, 27:493. In the case where a sample contains no detectable virus, a statistical analysis may be performed based on Poisson distribution (International Conference On Harmonization (ICH) Topic Q5A, Pages: 24-25, 1999) to determine the theoretical maximum possible titer for that sample. These analyses will be described in detail in the final report. The test results are reported as the reduction of the virus titer due to treatment with test composition expressed as log 10.

The tests are acceptable for evaluation of the test results if:

a. Virus must be recovered from the neutralizer effectiveness/viral interference control (not exhibiting cytotoxicity).

b. Viral-induced CPE must be distinguishable from test composition induced toxicity.

c. Cell Viability Control must not exhibit viral-induced CPE or cytotoxicity.

### Results

The results are shown in tables 4-6 below:

TABLE 4
Titer Results - Human Coronavirus

| | | | | | |
|---|---|---|---|---|---|
| | | Titer ± 95% Cl | Volume | Volume | Viral Load |
| Test Agent | Contact Time | (Log₁₀TCID₅₀/mL) | (mL) | Correction^{a} | (Log₁₀TCID₅₀) |

| Virus Stock Titer Control | NA | 6.00 ± 0.35 | — | — | — |
|---|---|---|---|---|---|
| Virus Recovery Control | 5 Minutes | 4.75 ± 0.25 | 3 | 2 | 5.53 ± 0.25 |
| Cell Viability Control | NA | No virus was detected, cells remained viable; media were sterile | | | |
| Composition tested | 5 Minutes | ≦0.83* | 3 | 2 | ≦1.61 |
| (Lot. No. 526) | | | | | |
| Composition tested | 5 Minutes | ≦0.83* | 3 | 2 | ≦1.61 |
| (Lot. No. 704) | | | | | |

^{a}Volume correction accounts for the neutralization of the sample post contact time.
*No virus was detected; the theoretical titer was determined based on the Poisson distribution.

TABLE 5
Neutralizer Effectiveness and Cytotoxicity Related Controls -
Human Coronavirus

| | Neutralizer Effectiveness/ | Cytotoxicity |
|---|---|---|
| Dilution** | Viral Interference Control | Control |
| | Composition tested (Lot No. 526) | |
| 10⁻¹ | virus detected in all four wells | no cytotoxicity observed in all |
| | | 4 wells |
| 10⁻² | virus detected in all four wells | no cytotoxicity observed in all |
| | | 4 wells |
| 10⁻³ | virus detected in all four wells | no cytotoxicity observed in all |
| | | 4 wells |
| | Composition tested (Lot No. 704) | |
| 10⁻¹ | virus detected in all four wells | no cytotoxicity observed in all |
| | | 4 wells |
| 10⁻² | virus detected in all four wells | no cytotoxicity observed in all |
| | | 4 wells |
| 10⁻³ | virus detected in all four wells | no cytotoxicity observed in all |
| | | 4 wells |

**Dilution refers to the dilution ration from the post-neutralized sample

TABLE 6
Neutralizer Effectiveness and Cytotoxicity Related Controls -
Human Coronavirus

| | Initial Load | Output Load | Viral Reduction |
|---|---|---|---|
| Test Agent | (Log₁₀TCID₅₀) | (Log₁₀TCID₅₀) | (Log₁₀TCID₅₀) |
| Composition tested | 5.53 ± 0.25 | ≦1.61 | ≧3.92 ± 0.25 |
| (Lot No. 526) | | | |
| Composition tested | | ≦1.61 | ≧3.92 ± 0.25 |
| (Lot No. 704) | | | |

### Conclusions:

Compositions of the present invention were evaluated for the ability to inactivate Human Coronavirus. Test personnel performed the inactivation procedure using Human Coronavirus to spike the test agent solution. Samples were titrated by 50% tissue culture infectious dose (TCID50) endpoint assay using MRC-5 cells. When tested as described above, compositions of the invention inactivated Human Coronavirus when the challenge virus was exposed to the test agents for 5 minutes at 21° C. Table 6 reports the individual Log 10 virus reduction factor for the test agent treatment procedure. All of the controls met the criteria for a valid test. These conclusions are based on observed data.

### Example 3

### Virucidal Suspension Efficacy Test: Influenza Virus

Influenza virus-killing ability of anti-viral compositions of the invention was tested on new and aged samples.

The Influenza viruses used (allantoic fluid stocks frozen at −80° C.) were:

H1N1: A/California/04/2009

H3N2: A/Brisbane/10/2007

In sterile PBS and influenza virus plaque assay overlay.

### Test Procedures:

1. Prepare 6 well plates of MDCK cells such that they are confluent monolayers at the time of experiment.
2. Thaw stock influenza viruses (infected chick allantoic fluid) at room temperature, vortex lightly and centrifuge (12,000×g) for one minute to remove any particulates. Pool virus from 2 vials into one tube and mix by vortexing.
3. Prepare 1 tube containing 4.0 ml of the test composition to be tested and 4 tubes containing 2 ml of sterile distilled water. Serially transfer and mix 2.0 ml of the 100% Test composition into a tube of water (making 50% Test composition), then 2.0 ml of that into another tube of water (making 25% Test composition), then 2 ml of that into another tube of water (making 12.5% Test composition); discard 2.0 ml from the 12.5% tube. This results in 5 tubes containing 2 ml of 100%, 50%, 25%, 12.5% and 0% Test composition. From each of these tubes, prepare 2 tubes containing 0.9 ml, these duplicates will be spiked with each of the 2 different viruses.
4. Mix Test composition and virus stock: At time 0, pipet 0.1 ml of the pooled virus stock with 0.9 ml of each test solution and mix immediately by light vortexing. The virus-test solution mixtures are then incubated at room temperature for 5 minutes.
5. After the 5 minute incubation, lightly vortex each tube again, and prepare serial ten-fold dilutions of each of the 10 solutions to achieve dilutions of 10-1 and 10-6. Prepare dilutions in a 96 well plate by serial transfer of 30 ul into 270 μl of PBS, using a multichannel pipettor.
6. As soon as virus-product dilutions are finished, inoculate in duplicate onto wells containing MDCK cells (standard plaque assay technique) Immediately prior to inoculation, medium was dumped from the 6 well plates and cells were washed with 3 ml PBS. Each well was inoculated with 100 μl samples of virus dilutions. Allow virus to adsorb for one hour at 37° C. with occasional rocking, then add 2 milliliters of first overlay solutions per well return to the incubator. The overlay consists of Modified Eagles Medium, 0.5% agarose, 0.5% bovine serum albumin, 0.0025% NaH2CO3, 1 mg/L TPCK trypsin, 100,000 U/L penicillin, 50 mg/L streptomycin, 50 mg/L gentamicin and 2.5 mg/L amphotericin B.
7. Two days after inoculation of the cells, apply a second overlay to each of the plates; this overlay is identical to the first overlay but supplemented with 6.6 mg/L neutral red as a stain. Count plaques 4-5 hours later and again the following day.

Test Composition: HPUS Sambucus Nigrans MOTHER TINTURE, HPUS Eucalyptol Oil, HPUS Eucalyptus Globulus MOTHER TINCTURE, HPUS Mentholum (Crystals), Poloxamer 407, Sorbitol, Glycerin, Sodium Chloride, Citric Acid (anhydrous), Sodium Benzoate, Methyl Salicylate, Ethanol, Thymolum, Water, pH to 3.2.

Results are reported as the mean plaque-forming units of virus for each tube:

### A. Virus: Influenza A/California/05/2009 H1N1

Contact time: 5 minutes

| | Plaque-forming units per ml of original mixture | | | |
|---|---|---|---|---|
| Composition | 100% Drug | 50% Drug | 25% Drug | 12.5% Drug |
| Test | 150 | 13200 | 19500 | 1204000 |
| Composition | | | | |
| H₂O (control) | 1843000 | NA | NA | NA |
| Spray bottle | NA | <50 | 5025 | 43000 |

### B. Virus: Influenza A/Sydney/05/1997 H3N2

Contact time: 5 minutes

| | Plaque-forming units per ml of original mixture | | | |
|---|---|---|---|---|
| Composition | 100% Drug | 50% Drug | 25% Drug | 12.5% Drug |
| Test | <50 | 20500 | 21700 | 1630000 |
| Composition | | | | |
| H₂O (control) | 1500000 | NA | NA | NA |
| Citric Acid | <50 | 3950 | 6000 | 205000 |
| 20% ETOH | | | | |
| Spray bottle | NA | <50 | 950 | 17900 |

The foregoing examples of the present invention have been presented for purposes of illustration and description. Furthermore, these examples are not intended to limit the invention to the form disclosed herein. Consequently, variations and modifications commensurate with the teachings of the description of the invention, and the skill or knowledge of the relevant art, are within the scope of the present invention. The specific embodiments described in the examples provided herein are intended to further explain the best mode known for practicing the invention and to enable others skilled in the art to utilize the invention in such, or other, embodiments and with various modifications required by the particular applications or uses of the present invention. It is intended that the appended claims be construed to include alternative embodiments to the extent permitted by the prior art.

What is claimed is:

1-6. (canceled)

7. A composition for use in ameliorating a viral infection, the composition comprising eucalyptol, menthol, and elderberry extract.

8. The composition of claim 7, further comprising at least one compound selected from the group consisting of poloxamer 407, xyitol, sucrose, saccharin, sorbitol, glycerin, sodium benzoate, octoxynol-9, citric acid, sodium chloride, thymol, ethanol, and methyl salicylate.

9. The composition of claim 7, wherein the elderberry extract is sourced as a tincture made from elderberry plant leaves or flowers (HPUS Sambucus nigrans Mother Tincture).

10. The composition of claim 7, wherein the composition is formulated for administration as a nasal spray.

11. The composition of claim 7, wherein the viral infection is caused by influenza virus, rhinovirus, coronavirus, parainfluenza virus, and respiratory syncytical virus.

12. A method of ameliorating a viral infection, comprising administering to a subject in need thereof the antiviral composition of claim 1.

13. The method of claim 12, wherein the viral infection is caused by a virus selected from influenza virus, rhinovirus, coronavirus, parainfluenza virus, and respiratory syncytical virus.

14. The method of claim 12, wherein the viral infection is caused by an influenza virus.

15. The method of claim 12, wherein the composition is administered in a delivery system selected from a nebulizer, an atomizer, a nasal spray bottle or a dropper.

16. (canceled)

17. The method of claim 12, wherein the composition is administered by application to epithelial cells of the subject.

18. The method of claim 17, wherein the epithelial cells are respiratory epithelial cells, adenoid epithelial cells or bronchial epithelial cells.

19. The method of claim 12, wherein the composition is administered as a nasal spray.

20. The method of claim 12, wherein the composition is administered via an inhaler.

21. (canceled)

22. The method of claim 15, wherein the composition is administered from one to four times a day.

23-25. (canceled)

26. An antiviral composition formulated as a nasal spray consisting of citric acid, water and ethanol.

27. An antiviral composition comprising eucalyptol, menthol, elderberry extract, methyl salicylate, water, ethanol, xylitol, poloxamer 407, glycerin, sorbitol, sodium chloride, citric acid, sodium benzoate, and thymol.

28. (canceled)
